(19) United States
(12) Patent Application Publication
(10) Pub. No.: US 2017/0172910 A1
(43) Pub. Date: Jun. 22, 2017

(54) COSMETIC COMPOSITION FOR LIGHTENING THE SKIN

(71) Applicant: ARKEMA FRANCE, Colombes (FR)

(72) Inventor: Helena CHEMINET, Bernay (FR)

(73) Assignee: ARKEMA FRANCE, Colombes (FR)

(21) Appl. No.: 15/300,595

(22) PCT Filed: Mar. 31, 2015
(86) PCT No.: PCT/FR2015/050834

§ 371 Date: Sep. 29, 2016

(30) Foreign Application Priority Data
Mar. 31, 2014 (FR) 1452792

(51) Int. Cl. A61K 8/97; A61K 8/02; A61Q 19/02; A61K 8/88

(52) U.S. Cl. CPC A61K 8/97; A61K 8/88; A61K 8/0279; A61Q 19/02

(57) ABSTRACT

A composition including a support including porous polyamide particles, having a specific surface area BET greater than or equal to 8 m²/g, Glycyrrhiza glabra root extract as a cosmetic active substance impregnated on the support. Also, cosmetic products incorporating said composition, in particular for lightening the skin. The composition may be in the form of a powder.

### FIELD OF THE INVENTION

The present invention relates to a cosmetic composition for lightening the skin, to the process for producing same and to the use thereof.

### TECHNICAL BACKGROUND

The reduction of brown spots on the skin due to the sun or to aging constitutes an important objective of the cosmetics industry. Likewise, there is a very clear tendency toward lightening the skin, in particular of the face, in particular in Asia, Latin America and Africa.

Glabridin, the principal component of the hydrophobic fraction of licorice (Glycyrrhiza glabra) root extracts, is known for its lightening and depigmenting effects on the skin. Nevertheless, this substance is tricky to formulate in cosmetic products, in particular because of its solubility limited to certain alcoholic solvents.

Document WO 98/58628 relates to a cosmetic composition for lightening or whitening the skin. The composition comprises a whitening agent, an exfoliant, an adsorbent or absorbent particulate carrier for the exfoliant and a cosmetic base comprising a film-forming agent.

Document WO 2006/037665 discloses an anhydrous cosmetic composition comprising a pulverulent phase comprising polyamide particles, a fatty phase and at least one moisture-sensitive active compound. The uses targeted are for example the care of greasy skin or the bleaching of the skin.

Moreover, the applicant is aware of other documents disclosing combinations of particles and of active compounds.

Thus, document WO 96/21422 describes an anhydrous cosmetic composition comprising a polar active dermatological agent, an adsorbent or absorbent particulate carrier for this agent, and a hydrophobic cosmetic base.

Document US 2002/0176843 describes the use of polyamide particles as an anti-irritant agent, in particular in compositions comprising, moreover, an irritant agent.

Document EP 1 493 433 describes porous particles loaded with cosmetic or pharmaceutical active compounds.

Document FR 2 944 443 describes a process for producing particles of polyamide-based free powder comprising a cosmetic or pharmaceutical agent.

There is a need to provide various novel cosmetic compositions for lightening human skin and reducing brown spots or age spots on human skin, and to facilitate the production of such compositions.

### SUMMARY OF THE INVENTION

The invention relates firstly to a composition comprising:
a support comprising porous polyamide particles, having a BET specific surface area greater than or equal to 8 m²/g;
Glycyrrhiza glabra root extract as active cosmetic substance impregnated on the support.

According to one embodiment of the invention, the composition is in the form of a powder, preferably in the form of a non aggregated or agglomerated powder.

According to one embodiment of the invention, the Glycyrrhiza glabra root extract is combined with a solvent, the solvent preferably being an alcohol and more particularly preferably butylene glycol.

According to one embodiment of the invention, the composition comprises from 50% to 90% by weight of polyamide particles and from 10% to 50% by weight of active mixture, preferably from 70% to 80% by weight of polyamide particles and from 20% to 30% by weight of active mixture, relative to the total weight of the composition.

According to one embodiment, the polyamide of the particles comprises polyamide 12 units, preferably comprises a molar content of polyamide 12 units of from 50% to 100%, more particularly preferably from 80% to 100%, the polyamide particles preferably being chosen from particles of polyamide 12, of copolyamide 6/12, of copolyamide 8/12 and of copolyamide 10/12, and mixtures thereof.

According to one embodiment, the polyamide particles are obtained by anionic polymerization, optionally by seeding with an inorganic or organic filler, of lactams and/or lactones in solution and/or in suspension in an organic liquid.

According to one embodiment, the polyamide particles have an average volume diameter Dv50 of between 3 and 15 μm, preferably between 5 and 12 μm.

According to another embodiment of the invention, the BET specific surface area of the support is chosen in the range of from 8 to 30 m²/g, preferably from 8 to 20 m²/g and for example from 9 m²/g to 16 m²/g.

The invention also relates to a cosmetic product comprising the composition according to above in combination with one or more cosmetically acceptable carriers, additives and/or other active substances.

According to one embodiment, the cosmetic product is in the form of a lotion, a cream, a gel, a free powder, a pressed powder or a stick.

The invention also relates to the use of the composition above or of the cosmetic product above for lightening the skin, reducing age spots or brown spots on the skin and/or depigmenting the skin.

The invention also relates to a process for preparing the composition according to the invention, comprising:
adding, dropwise or by spraying, an active mixture in the liquid state comprising Glycyrrhiza glabra root extract to the polyamide particles;
stopping the addition of active mixture before the polyamide particles begin to agglomerate; and
maintaining the stirring, preferably for a period of at least 5 minutes.

The present invention makes it possible to meet the need expressed above. It provides more particularly cosmetic products for lightening the skin and reducing brown spots or age spots on the skin, the production of which is simplified.

This is accomplished by virtue of the combination of the Glycyrrhiza glabra root extract as active substance with a porous polyamide powder, of relatively high specific surface area. These two elements are combined in the form of a composition which can then be easily formulated in various cosmetic products. It is a ready-to-use composition which does not require prior solubilization.

The Glycyrrhiza glabra root extract contains in particular glabridin as principal component of its hydrophobic fraction, said glabridin being an active compound which is particularly advantageous for the desired effects.

Thus, the invention offers time saving and simplification for the formulation of emulsion-type products, and provides a novel technical solution for anhydrous products such as pressed powders, free powders, cast products or sticks.

According to certain embodiments, the diffusion of glabridin in the stratum corneum of the skin and/or the effectiveness of glabridin in lightening the skin or reducing the spots is increased by virtue of the invention.

### DESCRIPTION OF EMBODIMENTS OF THE INVENTION

The invention is now described in greater detail and in a non-limiting manner in the description which follows.

The proportions, percentages or ratios, unless otherwise specified, are by weight.

The invention provides a composition comprising porous polyamide particles as support and Glycyrrhiza glabra root extract as active cosmetic substance impregnated on the support.

The porosity of the particles is characterized by the specific surface area (also called SSA). The porous particles of the invention have an SSA measured according to the BET method of greater than or equal to 8 m²/g. The BET (Brunauer-Emmet-Teller) method is a method known to those skilled in the art. It is in particular described in The Journal of the American Chemical Society, vol. 60, page 309, February 1938, and corresponds to international standard ISO 5794/1 (annex D). The specific surface area measured according to the BET method corresponds to the total specific surface area, that is to say it includes the surface area formed by the pores.

The particles are advantageously in the form of particles of free powder, that is to say they are not grouped together in the form of agglomerates or aggregates.

In the composition of the invention, the polyamide particles advantageously have an average volume diameter Dv50 ranging from 3 to 15 μm, and preferably from 5 to 12 μm. This average volume diameter can be measured according to the usual techniques, for example by means of a Coulter Multisizer II particle sizer according to standard ISO 13319. On the basis of the particle size distribution, it is possible to determine the average diameter and also the particle size dispersion (standard deviation) which measures the narrowing often the distribution, with a standard deviation of between 1 and 3 μm, or even often less than 2 μm. This particle size range associated with the porosity of the particles allows good penetration of the active agent into the horny layer of the epidermis, by slow diffusion.

Advantageously, the polyamide particles are spheroidal, that is to say in the shape of a spheroid, which means: almost spherical solid.

The term “polyamide” used in the present application has a generic meaning, that is to say it simultaneously covers homopolyamides, copolyam ides and copolyesteram ides, and mixtures thereof.

The polyamides according to the invention correspond to the products of condensation of lactams, of amino acids and/or of diacids with diamines and, more generally, correspond to the polymers formed by units linked to one another by amide groups.

The polyamides according to the invention may also result from the copolymerization of lactam(s) with one or more lactone(s) resulting in copolyesteram ides as described in patent EP 1 172 396.

The term “monomer” in the present description of the copolyam ides should be taken as meaning “repeating unit”. The case where a repeating unit of the polyamide consists of the combination of a diacid with a diamine is particular. It is considered that it is the combination of a diamine and a diacid, that is to say the diamine-diacid couple (in equimolar amount), which corresponds to the monomer. This is explained by the fact that, individually, the diacid or the diamine is merely a structural unit, which is not sufficient on its own to polymerize. In the case where the particles according to the invention comprise at least two different monomers, they form a copolymer such as a copolyamide or else a copolyesteramide.

By way of example of lactams, mention may be made of those which have from 3 to 12 carbon atoms on the main ring and which can be substituted. Mention may be made for example of β,β-dimethylpropiolactam, α,α-dimethylpropiolactam, amylolactam, caprolactam, capryllactam, enantholactam, 2-pyrrolidone and lauryllactam.

By way of example of a diacid (or dicarboxylic acid), mention may be made of acids having between 4 and 18 carbon atoms. Mention may for example be made of adipic acid, sebacic acid, azelaic acid, suberic acid, isophthalic acid, butanedioic acid, 1,4-cyclohexyldicarboxylic acid, terephthalic acid, the sodium or lithium salt of sulphoisophthalic acid, dimerized fatty acids (these dimerized fatty acids have a dimer content of at least 98% and are preferably hydrogenated) and dodecanedioic acid HOOC-(CH₂)₁₀-COOH.

By way of example of diamines, mention may be made of aliphatic diamines having from 6 to 12 atoms. They may be saturated acrylic and/or cyclic. By way of example, mention may be made of hexamethylenediamine, piperazine, tetramethylenediamine, octamethylenediamine, decamethylenediamine, dodecamethylenediamine, 1,5-diaminohexane, 2,2,4-trimethy-1,6-diaminohexane, diamine polyols, isophorone diamine (IPD), methyl pentamethylenediamine (MPDM), bis(aminocyclohexyl)methane (BACM), bis(3-methyl-4-aminocyclohexyl)methane (BMACM), meta-xylyenediamine, bis-p-aminocyclohexylmethane and trimethylhexamethylenediamine.

By way of example of amino acids, mention may be made of alpha, omega-amino acids, such as aminocaproic acid, 7-aminoheptanoic acid, 11-aminoundecanoic acid, n-heptyl-11-aminoundecanoic acid and 12-aminododecanoic acid.

By way of example of lactones, mention may be made of caprolactone, valerolactone and butyrolactone.

The monomers preferentially used in the invention are chosen from lactams such as, for example, lauryllactam, caprolactam, enantholactam, capryllactam or mixtures thereof. Preferably, lauryllactam is used alone or as a mixture with caprolactam.

Preferably, the polyamide particles according to the invention comprise, before impregnation, a molar percentage content of polyamide 12 included in the range of from 50% to 100%, preferably from 80% to 100%.

According to one embodiment, the polyamide particles are polyamide 12 particles, or copolyamide 6/12 particles, or copolyamide 8/12 particles, or copolyamide 10/12 particles. Preferably, they are polyamide 12 particles and/or copolyamide 6/12 particles.

Preferably, said polyamide-based powder particles are obtained at least partly by anionic polymerization, optionally by seeding with an inorganic or organic filler, of lactam(s) and/or lactone(s) in solution and/or in suspension in an organic liquid. Reference may be made, for example, to the processes described in documents EP 0 192 515 and FR 2 910 900. The polyamide particles can correspond to the powders sold under the trade name Orgasol® by Arkema.

One advantage of using polyamide particles which are made of polyamide 12 or contain predominantly polyamide 12 units is the very high compatibility of this polymer with the constituents of the surface of the skin. The molecular structure of the polyamide 12 chains is in several respects similar to that of ceramides, cholesterol and fatty acids, which are main constituents of the upper layers of the epidermis.

The polyamide 12 chains contain hydrocarbon-based units comprising 12 carbon atoms, forming lipophilic fatty chains similar to those of the lipid constituents of the skin. They thus provide great compatibility with the skin: softness, lack of irritation, excellent persistence. Finally, the amide functions of the polyamide 12 chains are polar functions which establish hydrogen bonds with the amide functions of the ceramides of the skin, reinforce the compatibility between particles of the invention and the surface of the skin, and extend adhesion of the particles to the surface of the skin. This makes it possible to extend the contact between the particles loaded with the active agent and the surface of the skin and optimizes the effectiveness of the agent at the surface of the skin.

The Glycyrrhiza glabra (licorice) root extract used in the invention contains in particular glabridin as active agent, that is to say the compound having the formula below:

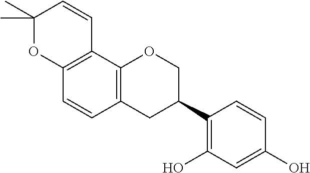 [SMILES: CC1(C)C=Cc2c(ccc3c2OC[C@@H](c2ccc(O)cc2O)C3)O1]

The Glycyrrhiza glabra root extract is provided in combination with a solvent, thus forming an “active mixture”.

The solvent is preferably an alcohol, more particularly preferably an alcohol of plant origin, and in particular butylene glycol. Ethanol, ethylene glycol, propylene glycol and mixtures of various alcohols may also be used.

The weight concentration of Glycyrrhiza glabra root extract in the solvent may be for example from 0.1% to 20%, in particular from 0.5% to 12%, or from 1% to 10%, and for example approximately 3% to 8%.

In the composition according to the invention, the weight proportion of polyamide particles may be from 50% to 90% and the weight proportion of active mixture may be from 10% to 50%; preferably, the weight proportion of polyamide particles may be from 70% to 80% and the weight proportion of active mixture may be from 20% to 30%.

Preferably, the weight proportion of glabridin in the composition according to the invention may be from 0.05% to 1%, and in particular from 0.1% to 0.5%. The glabridin may be quantitatively determined by high performance liquid chromatography (HPLC).

The composition according to the invention may consist essentially of, or even consist of, the polyamide particles and the active mixture described above. Alternatively, this composition may comprise various additives, such as pigments or preservatives.

This composition may also comprise one or more other active substances, in particular cosmetically active substances, such as, for example, other substances which promote lightening of the skin or reduction of skin spots. These active substances may in particular comprise kojic acid, ellagic acid, arbutin and derivatives thereof, hydroquinone, aminophenol derivatives, in particular N-cholesteryloxycarbonyl-para-aminophenol and N-ethyloxycarbonyl-para-aminophenol, iminophenol derivatives, L-2-oxothiazolidine-4-carboxylic acid or procysteine, and salts and esters thereof, calcium D-pantetheine sulfonate, arbutin and the other derivatives of Glycyrrhiza glabra, ascorbic acid and derivatives thereof, in particular ascorbyl glucoside, and plant extracts such as blackberry, skullcap and Bacopa monnieri extracts.

According to one particular embodiment, the composition above is free of fatty phase (such as in particular oils and/or waxes and more generally a phase which is fatty at the temperature of 25° C. and which consists of compounds without surfactant properties within the meaning of the McCutcheon and CTFA dictionaries).

The composition according to the invention can be produced by bringing the polyamide particles into contact with the active mixture described above. In particular, it is possible to do this by:
adding, dropwise or by spraying, the active mixture (in the liquid state) to the polyamide particles which are in free powder form, with stirring;
stopping the addition before the powder particles begin to agglomerate;
maintaining the stirring for at least 5 minutes, preferably at least 20 minutes; then
recovering the obtained free powder impregnated with the active mixture.

Document FR 2 944 443 provides examples of this type of process. Reference is made in particular (by analogy) to example 1 of said document.

The composition according to the invention can be incorporated into a cosmetic product (or cosmetic composition), in particular of the lotion, cream, gel, water/oil or oil/water emulsion (the oil being a silicone or non-silicone oil), free powder, pressed powder (or compact powder), cast product (foundation, lipstick, etc.) or stick type. The composition according to the invention can be incorporated into or mixed with a deodorant or antiperspirant product.

The cosmetic product may be a concealer product, or a product for making up the body and/or the face, or a foundation or else a face or body care product.

The cosmetic product may comprise, in addition to the composition according to the invention, one or more carriers compatible with a cosmetic use, in particular of liquid type, such as water, an aqueous-alcoholic solvent, one or more oils, or a combination thereof. By way of oil, use may be made of a synthetic oil or a natural oil, of plant or animal origin. One or more waxes (synthetic or plant or animal waxes) may also be included.

It may also comprise one or more additional active substances (in particular cosmetically active substances). It may also comprise fillers, for example talc, cellulose or fluorphlogopite. It may also comprise additives such as thickeners (in particular xanthan gum), surfactants, dyes, pigments, fragrances, preservatives, physical and chemical sunscreens, sequestrant agents, moisturizers such as polyols and in particular glycerol, pH adjusters (acids and/or bases), or antioxidants.

According to one embodiment, the cosmetic product is free of white pigments.

The cosmetic product may nevertheless comprise nanoparticles as sunscreen.

The cosmetic product according to the invention may be produced conventionally, by simply mixing the various compounds.

The composition according to the invention may be, for example, present in a weight proportion of from 0.1% to 20% in the cosmetic product, preferably from 0.5% to 15%, more particularly preferably from 1% to 10%, in particular from 2% to 8%, and for example from approximately 3% to 5%.

The composition according to the invention, just like the cosmetic products prepared therefrom, can be used to obtain a uniform and radiant complexion, lightening of the skin, depigmentation of the skin, or reduction or prevention of brown spots or age spots on the skin.

Preferably, the composition according to the invention, just like the cosmetic products prepared therefrom, offers long-term persistence.

Preferably, glabridin is used in the invention so as to inhibit tyrosinase activity and/or inhibit superoxide anion production and/or inhibit cyclooxygenase activity.

The composition of the invention also provides a “soft focus” effect on the skin, a sebum absorption effect, and a binding or compacting effect (in the case of a product in the form of a compact powder) and it has an improved sensory profile.

### EXAMPLES

The following examples illustrate the invention without limiting it.

### Example 1

### Cosmetic Product Formulations

A formulation A for a cosmetic cream, of the oil-in-water type, for lightening the skin, having the following weight composition is prepared:
Water: 83.44%.
Chlorophenesin: 0.28%.
Xanthan gum: 0.2%.
Hydroxyethyl acrylate and acryloyl taurate/sodium dimethyl copolymer: 0.5%.
Arachidyl alcohol and behenyl alcohol and arachidyl glucoside: 3.0%.
Caprylic/capric triglycerides: 5.0%.
Cyclohexasiloxane: 1.0%.
Phenoxyethanol and ethylhexylglycerol: 0.5%.
Antioxidant: 0.08%.
Glycerol: 3.0%.
Composition according to the invention: 3.0%.

A formulation B for a cosmetic stick for reducing brown spots on the skin, having the following weight composition, is prepared:
Octyldodecanol: 21.1%.
Jojoba esters: 13.0%.
Ozokerite: 2.0%.
Cera alba: 8.9%.
Caprylic/capric triglycerides: 4.1%.
Isostearyl isostearate: 10.5%.
Pentaerythrityl tetracaprylate/caprate: 35.6%.
Antioxidant: 0.05%.
Dimethicone/disodium stearoyl glutamate/aluminum hydroxide: 1.5%.
Synthetic fluorphlogopite, titanium dioxide: 0.25%.
Composition according to the invention: 3.0%.

A formulation C for a pressed cosmetic powder for lightening the skin and smoothing out imperfections, having the following weight composition, is prepared:
Octyldodecyl xyloside: 5.0%.
Isostearyl isostearate: 3.0%.
Talc: 44.7%.
Mica: 30.0%.
Talc and disodium stearoyl glutamate/aluminum hydroxide: 5.0%.
Cellulose: 5.0%.
Salicylic acid: 0.2%.
Pigments: 2.1%.
Composition according to the invention: 5.0%.

A formulation D for an alcoholic lotion for lightening the skin, intended to be applied after cleansing the skin and before optional application of a cream, is prepared:
1,3-butylene glycol: 7.00%.
Alcohol: 8.00%.
PEG-40 hydrogenated castor oil: 1.00%.
Polyacrylate crosspolymer-6: 0.10%.
Xanthan gum: 0.10%.
Cellulose: 5.0%.
Disodium EDTA: 0.05%.
Water: 79.15%.
PEG/PPG-17/6 copolymer: 3.00%.
Phenoxyethanol, methylisothiazolinone: 0.60%.
Composition according to the invention: 1.0%.

In the three formulations above, the composition according to the invention is produced from copolyamide 6/12 particles (75% by weight) and Glycyrrhiza glabra root extract in butylene glycol (25%).

The particles have an average diameter Dv50 of 10 μm.

The composition has a pH of 5 to 9, for example of 7.5.

### Example 2

### Effectiveness Tests

Formulation A of example 1 is tested on subjects for reduction of the visibility of brown spots on the skin, in comparison with a control formulation A′ identical to formulation A with the exception of the composition of the invention, which is replaced with 3% of water.

The formulations were tested by daily application by volunteers, with the formulation A on one half of the face and formulation A′ on the other half of the face and also on the hands. The volunteers were twenty women with spots on the face and on the hands.

The effectiveness on reduction of the visibility of brown spots is evaluated in three different ways: by image analysis, by measurement using a chromameter, and by clinical evaluation. The evaluation is performed at 28 days and at 56 days after the start of the treatment.

The results are summarized in the table below (as variation compared with the initial values at the start of the treatment):

| | Formulation A | Formulation A' |
|---|---|---|
| | (invention) | (control) |
| Visibility of the spots determined by | −14.3% | +4.2% |
| image analysis at 28 days | | |
| Visibility of the spots determined by | −18.8% | +3.2% |
| image analysis at 56 days | | |
| Intensity of the spots determined by | +17.8% | −1% |
| chromameter at 28 days | | |
| Intensity of the spots determined by | +30% | −2.5% |
| chromameter at 56 days | | |
| Subjects exhibiting reduced | +35% | +5% |
| pigmentation at 28 days (clinical | | |
| evaluation) | | |
| Subjects exhibiting reduced | +60% | +5% |
| pigmentation at 56 days (clinical | | |
| evaluation) | | |

With regard to the evaluation of the intensity of the spots by chromameter, it should be specified that the parameter indicated is the ITA angle. A high value corresponds to a low spot intensity and vice versa.

1. A composition comprising:
a support comprising porous polyamide particles, having a BET specific surface area greater than or equal to 8 m²/g;
Glycyrrhiza glabra root extract as active cosmetic substance impregnated on the support.

2. The composition as claimed in claim 1, which is in the form of a powder.

3. The composition as claimed in claim 1, wherein the Glycyrrhiza glabra root extract is combined with a solvent.

4. The composition as claimed in claim 1, comprising from 50% to 90% by weight of polyamide particles and from 10% to 50% by weight of active mixture, relative to the total weight of the composition.

5. The composition as claimed in claim 1, wherein the polyamide of the particles comprises polyamide 12 units.

6. The composition as claimed in claim 1, wherein the polyamide particles are obtained by anionic polymerization, optionally by seeding with an inorganic or organic filler, of lactams and/or of lactones in solution and/or in suspension in an organic liquid.

7. The composition as claimed in claim 1, wherein the polyamide particles have an average volume diameter Dv50 ranging from 3 to 15 μm, preferably from 5 to 12 μm.

8. The composition as claimed in claim 1, wherein the polyamide particles have a BET specific surface area included in the range of from 8 to 30 m²/g.

9. A cosmetic product comprising the composition as claimed in claim 1, in combination with one or more cosmetically acceptable carriers, additives and/or other active substances.

10. The cosmetic product as claimed in claim 9, which is in the form of a lotion, a cream, a gel, a free powder, a pressed powder or a stick.

11. A method of lightening the skin comprising utilizing the composition as claimed in claim 1.

12. A process for preparing the composition as claimed in claim 1, comprising:
adding, dropwise or by spraying, an active mixture in the liquid state comprising Glycyrrhiza glabra root extract to the polyamide particles;
stopping the addition of active mixture before the polyamide particles begin to agglomerate; and
maintaining the stirring.

13. The composition as claimed in claim 1, wherein the Glycyrrhiza glabra root extract is combined with a solvent, the solvent being an alcohol.

14. The composition as claimed in claim 1, comprising from 70% to 80% by weight of polyamide particles and from 20% to 30% by weight of active mixture, relative to the total weight of the composition.

15. The composition as claimed in claim 1, wherein the polyamide of the particles are chosen from particles of polyamide 12, of copolyamide 6/12, of copolyamide 8/12 and of copolyamide 10/12, and mixtures thereof.

16. The composition as claimed in claim 1, wherein the polyamide particles have a BET specific surface area included in the range of from 9 to 16 m^{2/}g.
